(19) United States
(12) Patent Application Publication
(10) Pub. No.: US 2012/0111756 A1
(43) Pub. Date: May 10, 2012

(54) ENEMA FORMULATIONS

(75) Inventors: Adam Payne, Oklahoma City, OK (US); Dennis Hair, Oklahoma City, OK (US); Justin Briggs, Oklahoma City, OK (US); Richard Harty, Oklahoma City, OK (US); Sachin Malhorta, Oklahoma City, OK (US)

(73) Assignee: ALTHEUS THERAPEUTICS, INC., Oklahoma City, OK (US)

(21) Appl. No.: 13/140,426

(22) PCT Filed: Dec. 16, 2009
(86) PCT No.: PCT/US09/68179

§ 371 Date: Oct. 18, 2011

Related U.S. Application Data
(60) Provisional application No. 61/138,507, filed on Dec. 17, 2008.

(51) Int. Cl. B65D 85/00; A61K 31/606

(52) U.S. Cl. 206/524.1; 514/162

(57) ABSTRACT

Disclosed are enema solutions or suspensions comprising 5-aminosalicylic acid, or a pharmaceutically acceptable salt or ester thereof, and N-acetylcysteine, or a pharmaceutically acceptable salt or ester thereof, methods of preparing the same, and bottles and packages containing the same.

### RELATED APPLICATIONS

The present application claims priority to the U.S. Provisional Application Ser. No. 61/138,507, filed on Dec. 17, 2008, by Harty et al., and entitled “ENEMA FORMULATIONS,” the entire disclosure of which is incorporated herein by reference.

### FIELD OF THE INVENTION

The present invention is in the field of pharmaceutical compositions, and particularly in the field of formulations for rectal delivery of pharmaceutically active ingredients.

### BACKGROUND OF THE DISCLOSURE

It has been shown that rectal delivery of an anti-inflammatory compound, for example a derivative of salicylic acids, such as 5-aminosalicylic acid (the active ingredient in Mesalamine and Mesalazine), in combination with an antioxidant, such as N-acetylcysteine, directly to the colon exerts a synergistic effect in the treatment of inflammatory bowel diseases, i.e., ulcerative colitis and Crohn's Disease. The data showing the results of these experiments in animals is disclosed in U.S. Pat. No. 7,417,037 to Harty, which is disclosed herein in its entirety, including the drawings. It is desirable to formulate these combinations in solutions that can effectively deliver the therapeutic agents to the colon of an animal, such as human, dog, cat or horse.

### SUMMARY OF THE INVENTION

Disclosed herein are pharmaceutical compositions in solution or suspension form comprising: a therapeutically effective amount of 5-aminosalicylic acid, or a pharmaceutically acceptable salt or ester thereof; and a therapeutically effective amount of N-acetylcysteine, or a pharmaceutically acceptable salt or ester thereof

Also disclosed are pharmaceutical compositions in solution or suspension form comprising: a therapeutically effective amount of 5-aminosalicylic acid, or a pharmaceutically acceptable salt or ester thereof a therapeutically effective amount of N-acetylcysteine, or a pharmaceutically acceptable salt or ester thereof; a polymer; a chelating agent; a preservative; and an antioxidant.

In addition, disclosed herein are methods of preparing an enema solution or suspension, the method comprising:
combining a first polymer, a chelating agent, a preservative, and an antioxidant with deoxygenated water to form a first mixture,
mixing the first mixture in a high speed mixer to obtain a second mixture; adding a therapeutically effective amount of 5-aminosalicylic acid, or a pharmaceutically acceptable salt or ester thereof, and a therapeutically effective amount of N-acetylcysteine, or a pharmaceutically acceptable salt or ester thereof, to the second mixture; and
adding a second polymer to the second mixture to obtain an enema solution or suspension.

Furthermore, disclosed herein are packaged enema solutions or suspensions comprising: an enema solution or suspension comprising: a therapeutically effective amount of 5-aminosalicylic acid, or a pharmaceutically acceptable salt or ester thereof; a therapeutically effective amount of N-acetylcysteine, or a pharmaceutically acceptable salt or ester thereof; a polymer; a chelating agent; a preservative; and an antioxidant; the enema solution or suspension contained in a bottle under an inert atmosphere, the bottle packaged in a diffusion-tight light-impervious package in the same inert gas as is present in the bottle.

### DETAILED DESCRIPTION OF THE EMBODIMENTS

The present inventors have discovered unique formulations in the form of a solution or a suspension that affect the efficient and convenient delivery of a therapeutic dose of a combination of a non-steroidal anti-inflammatory compounds, such as 5-aminosalicylic acid (5-ASA), or a pharmaceutically acceptable salt or ester thereof, and an antioxidant, such as N-acetylcysteine (NAC), or a pharmaceutically acceptable salt or ester thereof, to the colon of a subject.

The term “subject” refers to an animal, preferably a mammal, and most preferably a human, who is the object of treatment, observation or experiment. The mammal may be selected from the group consisting of mice, rats, rabbits, guinea pigs, dogs, cats, sheep, goats, cows, horses, primates, such as monkeys, chimpanzees, and apes, and humans. In some preferred embodiments, the mammal is a human, dog, cat or horse.

Thus, in the first aspect, disclosed herein are pharmaceutical compositions in solution or suspension form comprising:
a therapeutically effective amount of 5-aminosalicylic acid, or a pharmaceutically acceptable salt or ester thereof; and
a therapeutically effective amount of N-acetylcysteine, or a pharmaceutically acceptable salt or ester thereof.

By “solution” it is meant that all of the components in the mixture are uniformly distributed throughout the solvent such that there is a molecular interaction between the solvent molecules and the solute molecules. A “suspension,” on the other hand, refers to a mixture where the components within the mixture form aggregates, where aggregates are uniformly distributed throughout the mixture. A suspension can be an emulsion, as that term is understood in the art.

The term “pharmaceutically acceptable salt” refers to a formulation of a compound that does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

Throughout the present disclosure the term “about” a certain value means that a range of value±10%, and preferably a range of value±5%, is contemplated. Thus, for example, “about 100 mg” means that the contemplated value is between 90 mg and 110 mg, and preferably between 95 mg and 105 mg.

In some embodiments, the 5-aminosalicylic acid, or a pharmaceutically acceptable salt or ester thereof, is present at a dose in the range of 0.1-200 mg/mL. In other embodiments, the 5-aminosalicylic acid, or a pharmaceutically acceptable salt or ester thereof, is present at a dose in the range of about 1-150 mg/mL, or at a dose in the range of about 10-100 mg/mL, or at a dose in the range of about 30-100 mg/mL, or at a dose in the range of about 40-100 mg/mL, or at a dose in the range of about 50-100 mg/mL, or at a dose in the range of about 50-80 mg/mL, or at a dose in the range of about 50-75 mg/mL, or at a dose in the range of about 16-75 mg/mL. In certain embodiments, the 5-aminosalicylic acid, or a pharmaceutically acceptable salt or ester thereof, is present at a dose of about 66.7 mg/mL. In certain embodiments, the 5-aminosalicylic acid, or a pharmaceutically acceptable salt or ester thereof, is present at a dose of about 33.4 mg/mL. In certain embodiments, the 5-aminosalicylic acid, or a pharmaceutically acceptable salt or ester thereof, is present at a dose of about 16.7 mg/mL.

In some embodiments, the N-acetylcysteine, or a pharmaceutically acceptable salt or ester thereof, is present at a dose in the range of about 0.1-200 mg/mL. In other embodiments, the N-acetylcysteine, or a pharmaceutically acceptable salt or ester thereof, is present at a dose in the range of about 1-150 mg/mL, or at a dose in the range of about 5-100 mg/mL, or at a dose in the range of about 5-75 mg/mL, or at a dose in the range of about 5-60 mg/mL, or at a dose in the range of about 8-60 mg/mL, or at a dose in the range of about 8-50 mg/mL, or at a dose in the range of about 8-40 mg/mL. In certain embodiments, the N-acetylcysteine, or a pharmaceutically acceptable salt or ester thereof, is present at a dose of about 33.3 mg/mL. In certain embodiments, the N-acetylcysteine, or a pharmaceutically acceptable salt or ester thereof, is present at a dose of about 16.7 mg/mL. In certain embodiments, the N-acetylcysteine, or a pharmaceutically acceptable salt or ester thereof, is present at a dose of about 8.3 mg/mL.

In some embodiments, the pharmaceutical compositions described herein further comprise a polymer. In other embodiments, the polymer is a polymeric sugar. Polymeric sugars, or polysaccharides, include those sugars that are linked together through α-1,4 glycosidic bonds or β-1,4 glycosidic bonds. Examples of polymeric sugars include starch, glycogen, and cellulose. In some embodiments, the polymer is cellulose. In other embodiments, the cellulose is derivatized. In certain embodiments, the cellulose is microcrystalline cellulose, hydroxyethyl cellulose, or a combination thereof.

In some embodiments, the polymer is hydroxyethyl cellulose, which is present in between about 0.1% to about 40% weight/volume. In other embodiments, the hydroxyethyl cellulose is present in between about 0.1% to about 20% weight/volume, or between about 0.5% to about 15% weight/volume, or between about 0.5% to about 10% weight/volume, or between about 0.5% to about 5% weight/volume, or between about 0.9% to about 1.5% weight/volume. In certain embodiments, the hydroxyethyl cellulose is present in about 1.1% weight/volume. In certain embodiments, the hydroxyethyl cellulose is present in about 1.3% weight/volume. In certain embodiments, the hydroxyethyl cellulose is present in about 1.4% weight/volume. In certain embodiments, the hydroxyethyl cellulose is present in about 0.9% weight/volume.

In some embodiments, the polymer is microcrystalline cellulose, which is present in between about 0.1% to about 40% weight/volume. In other embodiments, the microcrystalline cellulose is present in between about 0.1% to about 20% weight/volume, or between about 0.3% to about 15% weight/volume, or between about 0.3% to about 10% weight/volume, or between about 0.5% to about 5% weight/volume, or between about 0.5% to about 1.5% weight/volume. In certain embodiments, the microcrystalline cellulose is present in about 0.58% weight/volume. In certain embodiments, the microcrystalline cellulose is present in about 0.85% weight/volume. In certain embodiments, the microcrystalline cellulose is present in about 0.93% weight/volume. In certain embodiments, the microcrystalline cellulose is present in about 1.4% weight/volume.

In some embodiments both hydroxyethyl cellulose (HEC) and microcrystalline cellulose (AVICEL® PH105) are present in the compositions described herein.

In some embodiments, the pharmaceutical compositions described herein further comprise a chelating agent. In certain embodiments, the chelating agent is a polymethylene diaminetetraacetic acid, or a pharmaceutically acceptable salt thereof. In some embodiments, the chelating agent is ethylene diaminetetraacetic acid, or a pharmaceutically acceptable salt thereof.

In some embodiments, the chelating agent is present in between about 0.01% to about 4% weight/volume. In other embodiments, the chelating agent is present in between about 0.01% to about 2% weight/volume, or between about 0.03% to about 1.5% weight/volume, or between about 0.03% to about 1.0% weight/volume, or between about 0.05% to about 0.5% weight/volume, or between about 0.05% to about 0.15% weight/volume. In certain embodiments, the chelating agent is present in about 0.07% weight/volume. In certain embodiments, the chelating agent is present in about 0.1% weight/volume. In certain embodiments, the chelating agent is present in about 0.13% weight/volume.

In some embodiments, the pharmaceutical compositions described herein further comprise a preservative. In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C₁-C₈ alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

As used herein, “Cₘ-Cₙ” in which “m” and “n” are integers refers to the number of carbon atoms in an alkyl group. That is, the alkyl can contain from “m” to “n”, inclusive, carbon atoms. Thus, for example, a “C₁-C₄ alkyl” group refers to all alkyl groups having from 1 to 4 carbons, that is, CH₃—, CH₃CH₂—, CH₃CH₂CH₂—, CH₃CH(CH₃)—, CH₃CH₂CH₂CH₂—, CH₃CH₂CH(CH₃)—, and (CH₃)₃CH—. If no “m” and “n” are designated with regard to an alkyl group, the broadest range described in these definitions is to be assumed.

In some embodiments, the alkyl is selected from the group consisting of methyl, ethyl, propyl, n-butyl, sec-butyl, and tert-butyl.

In some embodiments, the preservative is methylparaben, which is present in between about 0.01% to about 4% weight/volume. In other embodiments, the methylparaben is present in between about 0.01% to about 2% weight/volume, or between about 0.03% to about 1.5% weight/volume, or between about 0.03% to about 1.0% weight/volume, or between about 0.05% to about 0.5% weight/volume, or between about 0.09% to about 0.3% weight/volume. In certain embodiments, the methylparaben is present in about 0.1% weight/volume. In certain embodiments, the methylparaben is present in about 0.15% weight/volume. In certain embodiments, the methylparaben is present in about 0.2% weight/volume. In certain embodiments, the methylparaben is present in about 0.25% weight/volume.

In some embodiments, the preservative is propylparaben, which is present in between about 0.01% to about 4% weight/volume. In other embodiments, the propylparaben is present in between about 0.01% to about 2% weight/volume, or between about 0.01% to about 0.15% weight/volume, or between about 0.01% to about 0.10% weight/volume, or between about 0.01% to about 0.05% weight/volume. In certain embodiments, the propylparaben is present in about 0.015% weight/volume. In certain embodiments, the propylparaben is present in about 0.025% weight/volume. In certain embodiments, the propylparaben is present in about 0.035% weight/volume. In certain embodiments, the propylparaben is present in about 0.045% weight/volume.

In some embodiments both methylparaben and propylparaben are present in the compositions described herein.

In some embodiments, the pharmaceutical compositions described herein further comprise an antioxidant. In some embodiments, the antioxidant is selected from the group consisting of ascorbic acid, riboflavin, fumaric acid, malic acid, sodium ascorbate, sodium metabisulfite, propyl gallate, tocopherols, ascorbic acid palmitate, tertiary butylhydroquinone, butylated hydroxyanisole, sodium pyrosulfite, potassium pyrosulfite, and butylated hydroxytoluene. In certain embodiments, the antioxidant is ascorbic acid.

In some embodiments, the antioxidant is present in between about 0.01% to about 4% weight/volume. In other embodiments, the antioxidant is present in between about 0.01% to about 2% weight/volume, or between about 0.03% to about 1.5% weight/volume, or between about 0.03% to about 1.0% weight/volume, or between about 0.05% to about 0.5% weight/volume, or between about 0.09% to about 0.3% weight/volume. In certain embodiments, the antioxidant is present in about 0.1% weight/volume. In certain embodiments, the antioxidant is present in about 0.2% weight/volume.

In some embodiments, the pH of the solution or suspension is maintained at less than about 7.5. In other embodiments, the pH of the solution or suspension is maintained at less than about 6. In further embodiments, the pH of the solution or suspension is maintained at less than about 5.

In some embodiments, the pH of the solution or suspension is at a range of between about 5.5 to about 7.5. In other embodiments, the pH of the solution or suspension is at a range of between about 6.5 to about 6.8. In some embodiments, the pH of the solution or suspension is at about 6.6.

In some embodiments, the pH of the solution or suspension is adjusted by adding a base. Bases are compounds that when added to an aqueous solution, raise the pH of the solution. In some embodiments, the base is a physiologically acceptable base. In certain embodiments, the base is an alkali metal salt, such as a sodium or a potassium salt, an alkaline earth metal salt, such as a calcium or a magnesium salt, or an organic or inorganic compound containing a nitrogen or a phosphorous. In some embodiments, the base is selected from the group consisting of sodium hydroxide, potassium hydroxide, lye, calcium carbonate, sodium phosphate, sodium sulfate, sodium bisulfate, sodium sulfite, sodium bisulfate, and magnesium hydroxide. In some embodiments the base is sodium hydroxide.

In some embodiments, the pH of the solution or suspension is adjusted by adding an acid. Acids are compounds that when added to an aqueous solution, lower the pH of the solution. In some embodiments, the base is a physiologically acceptable base. In certain embodiments, an acid is selected from the group of hydrochloric acid, hydrobromic acid, hydroiodic acid, sulfuric acid, nitric acid, phosphoric acid, methanesulfonic acid, ethanesulfonic acid, p-toluenesulfonic acid, and salicylic acid. In some embodiments the acid is hydrochloric acid.

In some embodiments, both an acid and a base are added to the solution or suspension until the desired pH is reached.

In other embodiments, the pharmaceutical compositions described herein further comprise a physiologically acceptable buffer system. In certain embodiments, the buffer system is selected from the group consisting of phosphate buffer system, bicarbonate buffer system, and bisulfate buffer system.

In some embodiments, the solution or suspension is maintained under an inert atmosphere. In certain embodiments, the inert atmosphere comprises a gas selected from the group consisting of argon, nitrogen, and carbon dioxide. In some embodiments, the inert atmosphere comprises less than about 30 parts per million of oxygen.

In some embodiments, the solution or suspension comprises deoxygenated water. In certain embodiments, the deoxygenated water comprises less than about 30 parts per billion of oxygen.

In another aspect, disclosed herein are pharmaceutical compositions in solution or suspension form comprising:
a therapeutically effective amount of 5-aminosalicylic acid, or a pharmaceutically acceptable salt or ester thereof;
a therapeutically effective amount of N-acetylcysteine, or a pharmaceutically acceptable salt or ester thereof; and
one or more of a polymer; a chelating agent; a preservative; and an antioxidant;
   where the polymer, the chelating agent, the preservative, and the antioxidant are as described above.

In another aspect, disclosed herein are pharmaceutical compositions in solution or suspension form comprising:
a therapeutically effective amount of 5-aminosalicylic acid, or a pharmaceutically acceptable salt or ester thereof;
a therapeutically effective amount of N-acetylcysteine, or a pharmaceutically acceptable salt or ester thereof;
a polymer;
a chelating agent;
a preservative; and
an antioxidant;
   where the polymer, the chelating agent, the preservative, and the antioxidant are as described above.

In some embodiments, the solution or suspension comprising the 5-amino salicylic acid and the solution or suspension comprising the N-acetylcysteine are prepared and kept separately until shortly before their administration to the subject.

Thus, in another aspect, disclosed herein are pharmaceutical compositions in solution or suspension form comprising:
a therapeutically effective amount of 5-aminosalicylic acid, or a pharmaceutically acceptable salt or ester thereof; and
one or more of a polymer; a chelating agent; and a preservative;
   where the polymer, the chelating agent, and the preservative, are as described above.

In another aspect, disclosed herein are pharmaceutical compositions in solution or suspension form comprising:
a therapeutically effective amount of N-acetylcysteine, or a pharmaceutically acceptable salt or ester thereof; and
one or more of a polymer; a preservative; and an antioxidant;
   where the polymer, the preservative, and the antioxidant are as described above.

In another aspect, disclosed herein are pharmaceutical compositions in solution or suspension form comprising:
a therapeutically effective amount of 5-aminosalicylic acid, or a pharmaceutically acceptable salt or ester thereof; and
a polymer;
a chelating agent; and
a preservative;
   where the polymer, the chelating agent, and the preservative, are as described above.

In another aspect, disclosed herein are pharmaceutical compositions in solution or suspension form comprising:
a therapeutically effective amount of N-acetylcysteine, or a pharmaceutically acceptable salt or ester thereof;
a polymer;
a preservative; and
an antioxidant;
   where the polymer, the preservative, and the antioxidant are as described above.

In another aspect, disclosed herein are methods of preparing an enema solution or suspension, the method comprising:
combining a first polymer, a chelating agent, a preservative, and an antioxidant with deoxygenated water to form a first mixture,
mixing the first mixture in a high speed mixer to obtain a second mixture;
adding a therapeutically effective amount of 5-aminosalicylic acid, or a pharmaceutically acceptable salt or ester thereof, and a therapeutically effective amount of N-acetylcysteine, or a pharmaceutically acceptable salt or ester thereof, to the second mixture; and
adding a second polymer to the second mixture to obtain an enema solution or suspension.

The chelating agent, the preservative, and the antioxidant are as described above and are added in concentrations disclosed above. In some embodiments, the deoxygenated water is as described above.

In some embodiments, the first polymer, the chelating agent, the preservative, and the antioxidant are added at the same time to the deoxygenated water, whereas in other embodiments, these ingredients are added stepwise. In some of these embodiments, the solution is homogenized after the addition of each ingredient.

In some embodiments, the first polymer is cellulose, which can be microcrystalline cellulose. In certain embodiments, the microcrystalline cellulose is present in concentrations disclosed above. In some embodiments, the second polymer is cellulose, which can be derivatized cellulose, which can optionally be hydroxyethyl cellulose.

In some embodiments, the above method further comprises the step of adjusting the pH of the enema suspension or solution by the addition of a base or an acid. In other embodiments, the pH of the solution or suspension is adjusted by the addition of a physiologically acceptable buffer system.

In some embodiments, the method steps described above are carried out under an inert atmosphere. In certain embodiments, the inert atmosphere comprises a gas selected from the group consisting of argon, nitrogen, and carbon dioxide. In some embodiments, the inert atmosphere comprises less than 30 parts per million of oxygen.

In some embodiments, the mixer mixes the first mixture at a rate of between about 50-6000 rpm for about 15 minutes to about 24 hours. In certain embodiments, the mixing with the mixer produces an emulsion.

In some embodiments, the enema solution or suspension is poured into a bottle after it has been prepared by the above methods. In certain embodiments, the bottle is a plastic bottle, which can be a polyethylene bottle.

In some embodiments, the bottle is a single chamber bottle, which contains the suspension or solution. In other embodiments, the bottle is a multichamber bottle, where each chamber contains a separate mixture or solution. In some embodiments, one chamber holds a mixture or solution comprising the 5-ASA and various polymers and ingredients, such as a polymer, a chelating agent, a preservative, and the like. Another chamber holds a mixture or solution comprising NAC and various polymers and ingredients, such as a polymer, a preservative, an antioxidant and the like. In other embodiments, one chamber holds a solution of 5-ASA, one chamber holds a solution of NAC, and another one or more chambers hold a mixture of the various polymers and ingredients.

Prior to the administration to the subject, the contents of the various chambers are mixed to form a single mixture, which is then administered to the subject. Therefore, in some embodiments, the multichamber bottle comprises barriers between the various chambers that are easily removed, for example broken or perforated, to allow the ready mixing of the contents of the various mixtures. In other embodiments, the multichamber bottle comprises a mixing chamber, into which the contents of the various chambers flow for mixing prior to the administration to the patient.

In some embodiments, the enema solution or suspension is stored under an inert atmosphere in the bottle. In certain embodiments, the inert atmosphere comprises a gas selected from the group consisting of argon, nitrogen, and carbon dioxide. In some embodiments, the inert atmosphere comprises less than 30 parts per million of oxygen.

In further embodiments, the bottle is packaged in a diffusion-tight light-impervious package in the same inert gas as is present in the bottle. The diffusion-tight light-impervious package material is preferably made from a heat-sealable plastic-metal laminate, e.g., plastic-aluminum laminate, where any heat-sealable plastic material, e.g., polyethylene, might be used.

In another aspect, disclosed herein are packaged enema solutions or suspensions comprising:
an enema solution or suspension comprising:
   deoxygenated water;
   a therapeutically effective amount of 5-aminosalicylic acid, or a pharmaceutically acceptable salt or ester thereof;
   a therapeutically effective amount of N-acetylcysteine, or a pharmaceutically acceptable salt or ester thereof;
   a polymer;
   a chelating agent;
   a preservative; and
   an antioxidant;
      the enema solution or suspension contained in a bottle under an inert atmosphere, the bottle packaged in a diffusion-tight light-impervious package in the same inert gas as is present in the bottle.

The bottle, deoxygenated water, polymer, chelating agent, preservative, antioxidant, and inert atmosphere are as described above.

In some embodiments, the solution or suspension further comprises a physiologically acceptable buffer system, as described above.

### EXAMPLES

### Example 1

Purified water was deoxygenated to <10 ppm by purging the water container with highly purified compressed nitrogen. Hydroxy ethyl cellulose (HEC) was mixed with deoxygenated water with a mixer or homogenizer, until a clear first solution was obtained. Avicel, PH105 was mixed with deoxygenated water with a mixer or homogenizer; EDTA, methylparaben, propylparaben, and ascorbic acid were added to the mixture and the mixture was homogenized. 5-ASA and N-acetylcysteine were then added. The pH was adjusted by the addition of NaOH or HCl, as necessary. The HEC solution was added to the final mixture.

Final formula weight (% Weight/Vol) of materials: HEC (1.3%); Avicel (0.9%); EDTA (0.1%); Methylparaben (0.15%); Propylparaben (0.03%); Ascorbic acid (0.1%); 5-ASA (6.67%); NAC (3.33% or 1.66%); deoxygenated water (87.4%)

### Example 2

HEC, Avicel, EDTA, parabens, ascorbic acid, 5-ASA, and N-acetylcysteine were added to a container of deoxygenated water and mixed or homogenized mixture to obtain an enema suspension or solution. The pH was adjusted by the addition of NaOH or HCl, as necessary. The final formula weights were as in Example 1.

### Example 3

Hydroxy ethyl cellulose (HEC) was added to a container of deoxygenated water and the mixture was mixed a mixer or homogenizer, and heated until a clear first solution was obtained. Avicel PH105 was mixed with deoxygenated water in a separate container a mixer or homogenizer and heated. EDTA, methylparaben and propylparaben, and ascorbic acid were added and homogenized while heating. 5-ASA and N-acetylcysteine were then added and the pH was adjusted by the addition of NaOH or HCl, as necessary. The HEC solution was added to the mixture. The pH was adjusted again.

### Example 4

Avicel PH105 was mixed in a container with deoxygenated water using a mixer or homogenizer. EDTA, methylparaben and propylparaben, and ascorbic acid were added to the mixture and homogenized. 5-ASA and N-acetylcysteine were then added. Hydroxy ethyl cellulose (HEC) was then added to the mixture and the pH was adjusted by the addition of NaOH or HCl, as necessary.

1. A pharmaceutical composition in solution or suspension form comprising:
a therapeutically effective amount of 5-aminosalicylic acid, or a pharmaceutically acceptable salt or ester thereof; and
a therapeutically effective amount of N-acetylcysteine, or a pharmaceutically acceptable salt or ester thereof.

2. The pharmaceutical composition of claim 1, wherein the 5-aminosalicylic acid, or a pharmaceutically acceptable salt or ester thereof, is present at a dose in the range of 0.1-200 mg/mL.

3. The pharmaceutical composition of claim 1, wherein the N-acetylcysteine, or a pharmaceutically acceptable salt or ester thereof, is present at a dose in the range of 0.1-200 mg/mL.

4. The pharmaceutical composition of claim 1, further comprising a polymer, wherein the polymer is cellulose.

5. (canceled)

6. The pharmaceutical composition of claim 5, wherein the cellulose is derivatized, or wherein the cellulose is microcrystalline cellulose, hydroxyethyl cellulose, or a combination thereof.

7. (canceled)

8. The pharmaceutical composition of claim 7, wherein the hydroxyethyl cellulose is present in between about 0.1% to about 40% weight/volume, or wherein the microcrystalline cellulose is present in between about 0.1% to about 40% weight/volume.

9. (canceled)

10. The pharmaceutical composition of claim 1, further comprising an agent selected from the group consisting of:
i) a chelating agent, wherein the chelating agent is a polymethylene diaminetetraacetic acid, or a pharmaceutically acceptable salt thereof, or ethylene diaminetetraacetic acid, or a pharmaceutically acceptable salt thereof;
ii) a preservative, wherein the preservative is selected from the group consisting of a paraben, or a pharmaceutically acceptable salt thereof; methyl 4-hydroxybenzoate (methylparaben), or a pharmaceutically acceptable salt or ester thereof; and propyl 4-hydroxybenzoate (propylparaben), or a pharmaceutically acceptable salt or ester thereof; or a combination thereof; wherein the preservative is present in between about 0.01% to about 4% weight/volume; and
iii) a combination of i) and ii).

11. (canceled)

12. The pharmaceutical composition of claim 10, wherein the chelating agent is present in between about 0.01% to about 4% weight/volume.

13-14. (canceled)

15. The pharmaceutical composition of claim 10, wherein the paraben is an alkyl substituted 4-hydroxybenzoate, or a pharmaceutically acceptable salt or ester thereof.

16-17. (canceled)

18. The pharmaceutical composition of claim 1, further comprising an antioxidant, wherein the antioxidant is selected from the group consisting of ascorbic acid, propyl gallate, tocopherols, tertiary butylhydroquinone, butylated hydroxyanisole, sodium pyrosulfite, potassium pyrosulfite, and butylated hydroxytoluene.

19. (canceled)

20. The pharmaceutical composition of claim 18, wherein the antioxidant is present in between about 0.01% to about 4% weight/volume.

21. The pharmaceutical composition of claim 1, further comprising a physiologically acceptable buffer system, wherein the buffer system is selected from the group consisting of phosphate buffer system, bicarbonate buffer system, and bisulfate buffer system.

22. (canceled)

23. The pharmaceutical composition of claim 1, wherein the pH of the solution or suspension is at a range of between about 5.5 to about 7.5.

24. The pharmaceutical composition of claim 1, wherein
i) the solution or suspension is maintained under an inert atmosphere: and/or
ii) the solution or suspension comprises deoxygenated water.

25. (canceled)

26. A method of preparing an enema solution or suspension, the method comprising:
combining a first polymer, a chelating agent, a preservative, and an antioxidant with deoxygenated water to form a first mixture,
mixing the first mixture in a high speed mixer to obtain a second mixture;
adding a therapeutically effective amount of 5-aminosalicylic acid, or a pharmaceutically acceptable salt or ester thereof, and a therapeutically effective amount of N-acetylcysteine, or a pharmaceutically acceptable salt or ester thereof, to the second mixture; and
adding a second polymer to the second mixture to obtain an enema solution or suspension.

27. The method of claim 26, wherein:
i) the first polymer is cellulose;
ii) the chelating agent is a polymethylene diaminetetraacetic acid, ethylene diaminetetraacetic acid, or a pharmaceutically acceptable salt thereof;
iii) the preservative is a paraben, or a pharmaceutically acceptable salt thereof;
iv) the antioxidant is selected from the group consisting of ascorbic acid, propyl gallate, tocopherols, tertiary butylhydroquinone, butylated hydroxyanisole, sodium pyrosulfite, potassium pyrosulfite, and butylated hydroxytoluene; or
v) the second polymer is cellulose; or
vi) a combination of any two or more of i)-iv).

28-31. (canceled)

32. The method of claim 26, wherein the method steps are carried out under an inert atmosphere.

33. The method of claim 26, wherein the high speed mixer mixes the first mixture at a rate of between 50-6000 rpm for 15 minutes to 24 hours.

34. A packaged enema solution or suspension comprising:
an enema solution or suspension comprising:
deoxygenated water;
a therapeutically effective amount of 5-aminosalicylic acid, or a pharmaceutically acceptable salt or ester thereof;
a therapeutically effective amount of N-acetylcysteine, or a pharmaceutically acceptable salt or ester thereof;
a polymer;
a chelating agent;
a preservative; and
an antioxidant;
the enema solution or suspension contained in a bottle under an inert atmosphere, the bottle packaged in a diffusion-tight light-impervious package in the same inert gas as is present in the bottle.

35. The packaged solution or suspension of 34, wherein the bottle is:
i) a plastic bottle;
ii) a polyethylene bottle; or
iii) a multichamber bottle; or
iv) a combination of i)-iii).

36-37. (canceled)
